(19) United States
(12) Patent Application Publication
(10) Pub. No.: US 2008/0193549 A1
(43) Pub. Date: Aug. 14, 2008

(54) Hoki Fish Liver Oil, Fractions Thereof And Therapeutic Uses

(75) Inventor: Paul Frank Davis, Wellington (NZ)

Correspondence Address: SEED INTELLECTUAL PROPERTY LAW GROUP PLLC, 701 FIFTH AVE, SUITE 5400, SEATTLE, WA, 98104, US

(73) Assignee: Otago Innovation Limited, Dunedin (NZ)

(21) Appl. No.: 11/631,721

(22) PCT Filed: Jul. 5, 2005
(86) PCT No.: PCT/NZ05/00153

§ 371 Date: Feb. 4, 2008

(30) Foreign Application Priority Data
Jul. 5, 2004 (NZ) 533918

(51) Int. Cl. A61K 35/60; A61P 37/00; A61P 35/00; A61P 19/00

(52) U.S. Cl. 424/523

(57) ABSTRACT

Oil obtained from the liver of hoki fish is a potent inhibitor of angiogenesis and inflammation, and a modulator of the immune system. Compositions containing hoki liver oil, or fractions thereof, are useful for treating diseases or disorders associated with angiogenesis and inflammation. Certain oil fractions are potent stimulants of the immune system whereas other fractions are potent suppressants of the immune system.

### TECHNICAL FIELD

This invention relates to oil from the liver of hoki fish, or fractions of hoki liver oil, and the use of such oil or fractions in the prevention or treatment of diseases or disorders relating to angiogenesis or inflammation, or in the modulation of the immune system.

### BACKGROUND

It is becoming increasingly accepted that fish consumption is beneficial for the maintenance of good health. Much of this advantageous effect has been attributed to lipids present in fish, especially fatty acids. Fish meat and other tissues are commonly enriched in polyunsaturated fatty acids (PUFAs), particularly those designated as omega-3 (ω-3) PUFAs.

Hoki (Macruronus novaezelandiae) has levels of ω-3 PUFAs higher than found in many other marine organisms. In particular, the level of docosahexaenoic acid (DHA) in hoki is one of the highest known among marine species. The consumption of DHA, and of the related PUFA, eicosapentaenoic acid (EPA), is beneficial for the prevention of cardiovascular conditions, cancers, infections, inflammation and several psychological conditions.

However, it is likely that PUFAs are not the only biologically active constituents in fish. Other lipid components, such as phospholipids, triglycerides, sterols, and gangliosides, could also have beneficial health effects, either on their own or in combination with PUFAs. There are significant levels of these components in fish organs.

Liver oils from some marine species have been marketed as a general tonic with reputed biological activities in a number of health aspects. Among the well-known are shark liver oils and cod liver oils. From many species, the liver is waste but comprises a substantial portion of the mass of the fish.

In the ongoing search for natural products that have health benefits, the applicant investigated the ability of oil obtained from the liver of hoki, rather than from the muscle flesh of hoki, to inhibit angiogenesis or inflammation, or to modulate the immune system. The applicant surprisingly found that hoki liver oil exhibited not only one but several potent biological activities. The oil was active against both angiogenesis and inflammation, and certain oil fractions were potent stimulants of the immune system whereas other fractions were potent suppressants of the immune system.

It is therefore an object of the invention to provide hoki liver oil, or a fraction thereof, as an anti-angiogenesis agent, an anti-inflammatory agent, or an immuno-modulating agent, or to at least provide a useful choice.

### STATEMENTS OF INVENTION

In a first aspect of the invention, there is provided a composition containing hoki liver oil, or a fraction thereof, and a suitable carrier, diluent or excipient, which composition inhibits angiogenesis or inflammation, or modulates the immune system.

Preferably the composition inhibits both angiogenesis and inflammation. It is further preferred that the composition additionally modulates the immune system.

In a preferred embodiment of the invention, the composition contains a hoki liver oil fraction.

Preferably the fraction is a fraction that contains free fatty acids, particularly long chain free fatty acids of at least 20 carbon atoms.

The fraction is preferably enriched in at least one omega-3 polyunsaturated fatty acid (or ester thereof) relative to hoki liver oil.

The fraction preferably contains at least 5% (by weight of total free fatty acids) of eicosapentaenoic acid (EPA). Alternatively, the fraction preferably contains at least 15% (by weight of total free fatty acids) of docosahexaenoic acid (DHA).

The fraction is preferably enriched in triglycerides relative to hoki liver oil.

It is further preferred that the fraction contains both diglycerides and triglycerides.

In a preferred embodiment of the invention, the fraction contains no phospholipids.

In a second aspect of the invention, there is provided the use of hoki liver oil, or a fraction thereof, in the prevention or treatment of angiogenesis or inflammation, or in the modulation of the immune system.

Preferably the use is in the prevention or treatment of both angiogenesis and inflammation. More preferably the use is in the prevention or treatment of both angiogenesis and inflammation, and the simultaneous modulation of the immune system.

The angiogenesis may be associated with cancer, retinopathy, macula degeneration, arthritis or psoriasis. Cancers treatable are typically solid tumour cancers including melanoma, prostate cancer, brain cancer, colon cancer, lung cancer, and breast cancer. Arthritis includes both rheumatoid arthritis and osteoarthritis.

The inflammation may be associated with any inflammatory condition including inflammations of joints, lungs, skin and gut, as well as infections and cardiac conditions.

The modulation of the immune system may be stimulation of the immune system to treat conditions such as cancer, microbial infection, or toxicity. Alternatively, the modulation of the immune system may be suppression of the immune system to treat autoimmune diseases or prevent rejection following organ transplantation.

The invention also provides the use of hoki liver oil, or a fraction thereof, as an anti-angiogenesis agent, or an anti-inflammatory agent, or as an immuno-modulation agent.

The invention further provides the use of hoki liver oil, or a fraction thereof, in the preparation or manufacture of an anti-angiogenesis agent, or an anti-inflammatory agent, or an immuno-modulation agent.

The invention further provides a method of preventing or treating a disease or disorder associated with angiogenesis or inflammation, or modulation of the immune system, by administering to a human or non-human animal a therapeutically effective amount of hoki liver oil, or a fraction thereof.

### DETAILED DESCRIPTION

Hoki (Macruronus novaezealandiae) is New Zealand's most important commercial fish species. It lives mainly in the middle water depths and is taken by mid-water trawling, usually at depths of around 300-600 metres. Most hoki are between 60-100 cm long. Other names include blue hake, blue grenadier, whiting (incorrectly) and whiptail.

The oil extracted from the livers of hoki has been found by the applicant to exhibit several potent biological modulatory activities. It shows strong anti-inflammatory activity. It also suppresses superoxide production by activated neutrophils. Although there is increased inhibitory response with increasing doses of hoki liver oil, this increase does not follow a linear pattern. Additionally, when the oil is diluted 10-fold and re-assayed the inhibition remains but again the linearity of response is not strong.

Monocytes present in the white cell population of the circulating blood are recruited to sites of disease, damage and infection by inflammatory cytokines. They are converted to macrophages which produce modulators that enhance the inflammatory response. The applicant has determined that hoki liver oil and fractions of the oil have strong antagonistic activities in this conversion.

Cyclo-oxygenase (COX) enzymes are crucial in the metabolism of arachidonic acid. In particular, the COX-2 isoenzyme has been shown to have elevated activity in a number of inflammatory conditions. The COX-1 isoenzyme is a constitutive enzyme and inhibition of it can have undesirable effects especially in the gastrointestinal tract and the kidneys. The applicant has found that hoki liver oil has antagonistic activity on cyclo-oxygenases with the effect being stronger on the COX-2 isoenzyme.

One of the major features of a response of the immune system to a modulator is the alteration in the rate of proliferation of T-cells. Therefore it is of interest to investigate whether a modulator can alter the proliferation rate of T-cells that have already been stimulated by a mitogen such as Concanavalin A (Con A). The applicant has found that hoki liver oil has an inhibitory effect on the proliferation of T-cells especially when the culturing commences. However, the oil suppresses the effect of the Con A, especially at higher concentrations. If the oil is added after the stimulation of the proliferation has been well-established, the oil is able to significantly further stimulate the proliferation rate.

The applicant has additionally found that hoki liver oil is potently anti-angiogenic. Even at a 1000-fold dilution the oil completely inhibited the growth of new blood vessels in the aortic ring assay. Further dilution showed a dose response relationship with the antagonistic activity being lost at a dilution of 10000.

In summary, hoki liver oil has multiple biological modulatory activities. It has significant anti-inflammatory activities, including the inhibition of activated neutrophils, the conversion of monocytes to macrophages, and the modulation of the activity of the COX-2 enzyme. It can modulate T-cell immune response depending on the circumstances. It has strong anti-angiogenic activity.

The applicant established that certain fractions of hoki liver oil were more active than others. Fractions containing certain diglycerides and triglycerides and small amounts of sterols and monoglycerides/polar lipids were more active than other fractions. Fractions having lower amounts of 18:1 (n-9), but higher concentrations of EPA and DHA and other C22 unsaturated fatty acids, were the most active.

The oil and oil fractions, or compositions containing them, may be administered in any suitable form. Oral administration is the preferred administration method for most uses, although topical administration may be the preferred method for some uses such as skin infections.

The compositions of the invention may be formulated in a variety of ways. They may be formulated with other oils, such as olive oil. They may be encapsulated in a gelatin capsule or other similar capsule or coating. Taste or smell masking methods may also be employed in formulating the compositions of the invention.

The invention will be described further with reference to the following examples. It is to be appreciated that the invention is not limited in any way by or to these examples.

### EXAMPLES

### Example 1

### Samples Assayed

Samples of unfractionated hoki liver oil, as well as four fractions of hoki liver oil, were assayed. The hoki liver oil fractions were obtained by fractional distillation of the oil to produce fatty acid ethyl ester molecular distillates. The fractions are identified as Fractions 1 to 4. The composition of each sample was determined by thin layer chromatography (TLC) and by gas-chromatography (GC).

Non-polar lipids were analysed on silica gel plates using hexane:diethyl ether:acetic acid (80:20:1). Fraction 1 does not appear to contain triglycerides, diglycerides, free fatty acids or sterols. It appears to be pure fatty acid ethyl esters with a small quantity of monoglycerides. Fractions 2 and 3 are very similar. Both contain predominantly fatty acid ethyl esters. There are no triglycerides or diglycerides present, but small amounts of free fatty acids, sterols and monoglycerides. Fraction 4 contains significant levels of fatty acid ethyl esters, triglycerides, diglycerides, sterols and monoglycerides.

The analysis of the polar lipids showed that there were no phospholipids in any of the fractions. All the lipids present appear to be non-polar.

The analyses of the fatty acid content of each fraction are given in Table 1. Fraction 3 is the most enriched in long chain polyunsaturated fatty acids with 24% DHA. Fraction 4 has comparatively reduced levels of DHA and EPA.

TABLE 1
Fatty Acid Profiles of Samples (% of total)

| | | Fraction | Fraction | Fraction | Fraction |
|---|---|---|---|---|---|
| | Hoki Liver Oil | 1 | 2 | 3 | 4 |
| 14:0 | 3.81 | 6.82 | 2.19 | 0.40 | 2.48 |
| i15:0 | 0.29 | 0.46 | | | |
| 15:0 | 0.57 | 1.24 | 0.33 | | |
| i16:0 | | 0.37 | | | |
| 16:0 | 16.57 | 26.25 | 9.78 | 2.90 | 10.21 |
| 16:1 (n-7 | 5.77 | 9.75 | 3.61 | 1.38 | 3.64 |
| | 0.25 | 0.96 | 0.29 | | 0.35 |
| Phytanic + 16:2 | 1.26 | 1.50 | 0.71 | 0.39 | 0.75 |
| | 0.28 | 0.60 | 0.22 | | 0.34 |
| | | | 0.21 | | |
| 17:1 | 0.72 | 1.16 | 0.42 | 0.25 | 0.50 |
| | 0.27 | 0.32 | 0.22 | | 0.22 |
| | | | | | 0.33 |
| 18:0 | 2.75 | 2.93 | 2.26 | 1.98 | 2.49 |
| 18:1 (n-9) | 22.43 | 25.66 | 19.14 | 16.00 | 17.14 |
| 18:1 (n-7) | 3.24 | 3.77 | 2.90 | 2.38 | 3.22 |
| 18:1 (n-5) | 0.31 | 0.29 | 0.25 | | 0.25 |
| 18:2 (n-6) | 1.23 | 1.39 | 1.16 | 0.92 | 1.39 |
| | | | 0.27 | 0.27 | |
| 18:3(n-3) | 0.63 | 0.65 | 0.55 | 0.47 | 0.94 |
| 18:4(n-3) | 0.84 | 1.02 | 0.76 | 0.56 | 1.66 |
| | 0.58 | 0.46 | 0.79 | 1.00 | 0.41 |
| 20:1(n-9) | 7.53 | 3.11 | 12.51 | 14.89 | 7.50 |
| 20:1(n-7) | 0.32 | 0.26 | 0.49 | 0.59 | 0.37 |
| 20:2(n-6) | 0.36 | | 0.45 | 0.56 | 0.34 |
| 20:4(n-6) AA | 0.51 | 0.42 | 0.72 | 0.77 | 0.78 |
| 20:3(n-3) | 0.26 | | 0.32 | 0.41 | 0.31 |
| 20:4(n-3) | 1.60 | 0.57 | 2.23 | 2.56 | 2.02 |
| 20:5(n-3) EPA | 6.10 | 3.05 | 8.01 | 8.67 | 8.86 |
| 22:1 (n-11) | 2.98 | 0.56 | 4.17 | 6.67 | 4.78 |
| 22:1 (n-9) | 0.97 | 0.20 | 1.34 | 1.92 | 1.22 |
| 21;5(n-3) | | | 0.37 | 0.47 | 0.54 |
| 22:4(n-6) | 0.29 | | 0.31 | 0.46 | 0.33 |
| 22:5 (n-3) | 1.52 | 0.30 | 2.52 | 3.41 | 3.08 |
| | 0.71 | | | | |
| 22:6 (n-3) DHA | 10.63 | 2.28 | 16.70 | 23.91 | 18.54 |
| | | | | 0.31 | |
| 24:1 | 1.53 | | 1.20 | 2.22 | 1.60 |

### Example 2

### Angiogenesis Assays

Aliquots of lipid extracts were assayed for their ability to modulate angiogenesis using the rat aortic ring model. The assay used is based on the methods of Nicosia and Ottinetti (Lab Invest 63: 115-122 (1994)) and Brown et al (Lab Invest 75: 539-555 (1996)). Rings cut from a piece of cleaned rat aorta are placed between two layers of fibrin gel in wells of a 24 well culture plate (one ring per well). The gel is prepared in MCDB131 medium and overlaid with this medium. The culture (which is serum-free) is incubated at 37° C. in a 3% CO₂/97% air atmosphere. After approximately 5 days, microvessels can be detected growing from the surface of the ring. Digital images of each ring are recorded every 2 or 3 days and the area occupied by new microvessels relative to the size of the ring is calculated using NIH Image software. The rate of growth of vessels in each well is recorded. Each assay was performed in triplicate and the mean growth compared with that of a control set of wells which contained the solvent only. The hoki liver oil and its fractions were diluted with 20% ethanol. Each was assayed and the activity compared with that of 2% ethanol (equivalent concentration of the solvent).

TABLE 2
Effect on Angiogenesis

| | | Angiogenesis | % |
|---|---|---|---|
| | Dilution | (Units) | Inhibition |
| Unfractionated Oil | 1:1000 | 0 | 100 |
| | 1:5000 | 4.643 | 43.69 |
| | 1:10000 | 8.020 | 2.73 |
| Fraction 1 | 1:1000 | 2.559 | 68.96 |
| Fraction 2 | 1:1000 | 1.165 | 85.88 |
| Fraction 3 | 1:1000 | 0 | 100 |
| | 1:5000 | 3.395 | 55.82 |
| Fraction 4 | 1:1000 | 0 | 100 |
| | 1:5000 | 1.088 | 86.80 |
| | 1:10000 | 3.056 | 62.93 |
| Control | | 8.245 | |

The mid-point of the inhibitory activity of the unfractionated oil is about 1:4000 dilution. However, the fractions show differential activity. Fraction 1 and Fraction 2 are less inhibitory than the unfractionated oil. Fraction 3 and Fraction 4 are more inhibitory.

### Example 3

### Immune Modulation

A spleen was isolated from a rat and a T-cell rich fraction (free of red blood cells) was prepared by density gradient centrifugation. The cell preparation was diluted to about 1.2×10⁶ cells per ml with RPMI culture medium and approximately 2.4×10⁵ cells were added to each well of a 96 well culture plate. For each sample to be tested, 12 wells (a row) were used. Three were for the sample added at the start of the culture; three had the sample added 24 hrs after culturing commenced; three had the sample and the mitogen, Concanavalin A (Con A) (1 μg/ml), added at the start of the culture; and three had the Con A added at the start of the culture and the test sample added after 24 hrs incubation. The plate was incubated at 37° C. in 5% CO₂/95% air for 72 hrs. The incubation was terminated by adding 15 μl of MTT solution (5 mg/ml) to each well and the cells lysed 2 hrs later with 100 μl of 10% Sodium Dodecyl Sulphate/45% dimethylformamide, pH 4.7. The formazan crystals were dissolved by incubating overnight at 37° C. before reading the absorbance at 570 nm. The mean absorbance of each triplicate of wells was determined and this value was compared with the value obtained in the corresponding control wells. The effect on the proliferation of T cells derived from rat spleen in both the presence and absence of the mitogen, Concanavalin A, is summarised in Table 3.

TABLE 3
Effect on Proliferation Rate of Spleen Cells (% of control cultures)

| | Dilution | A | B | C | D |
|---|---|---|---|---|---|
| Unfractionated Oil | 1:2500 | 66.45 | 66.80 | 131.60 | 110.02 |
| | 1:5000 | 64.49 | 72.84 | 92.64 | 113.50 |
| | 1:10000 | 59.37 | 71.25 | 122.72 | 109.09 |
| Fraction 1 | 1:2500 | 60.02 | 63.26 | 101.35 | 92.84 |
| | 1:5000 | 60.68 | 70.18 | 113.62 | 92.40 |
| | 1:10000 | 78.21 | 78.91 | 115.46 | 98.54 |
| Fraction 2 | 1:2500 | 58.82 | 54.64 | 107.29 | 105.34 |
| | 1:5000 | 57.41 | 57.51 | 88.25 | 95.02 |
| | 1:10000 | 62.09 | 63.58 | 110.54 | 95.89 |
| Fraction 3 | 1:2500 | 59.59 | 55.17 | 21.38 | 56.42 |
| | 1:5000 | 61.87 | 71.57 | 81.56 | 78.51 |
| | 1:10000 | 55.77 | 58.57 | 86.27 | 98.91 |
| Fraction 4 | 1:2500 | 87.04 | 59.11 | 25.48 | 19.31 |
| | 1:5000 | 52.83 | 52.18 | 16.67 | 89.25 |
| | 1:10000 | 58.50 | 56.12 | 41.33 | 89.95 |

Column A is the effect of adding the sample at the same time as the culture is started.
Column B is the effect produced by adding the sample 24 hours after the culture is commenced.
Column C is the effect when the sample and the mitogen (Concanavalin A) are added simultaneously at the start of the culture. This effect is that produced in relation to the effect produced by Con A alone.
Column D is the effect noted when the sample is added 24 hours after the Con A has been added to the culture.

The unfractionated oil has an inhibitory effect when added to spleen cells. 30% inhibition was observed, and does not appear to be dose dependent. However, in the presence of Con A, there is a stimulation of the cell growth. The stimulation is greater if the oil is added at the same time as the Con A is added, but is less if the oil is added after the Con A has stimulated.

Fraction 1 inhibits the proliferation of the spleen cells. It seems to parallel the behaviour of the unfractionated oil both in the level of inhibition and the lack of dose response. Likewise, when Fraction 1 is added with Con A it is stimulatory, with the stronger stimulation being at the time of simultaneous addition. Fraction 2 behaves similarly to Fraction 1.

Fraction 3 is about 40% inhibitory. When added with Con A the highest concentration is strongly inhibitory causing nearly 80% inhibition, which means that it abolished the effect of the Con A as well as some of the innate proliferation. This effect is largely lost at the lower concentrations. When added after the Con A stimulation is established, the pattern is similar but not as severe.

Fraction 4 causes around 40% inhibition. There is little dose response effect observed. However, unlike the other fractions, Fraction 4 is very potently inhibitory of the stimulatory effect of the Con A. In fact, Fraction 4 even inhibits the innate growth rate. That is, Fraction 4 completely abolishes the effect of Con A and further inhibits the growth rate. The inhibition is stronger when it is added at the same time as the Con A. When added to cells already stimulated with Con A the effect is considerably less.

### Example 4

### Anti-Inflammatory Assays

(a) Neutrophil Activation

The in vitro anti-inflammatory screening assay used in this investigation involved determining the effect of a test material on activated neutrophils. The production of superoxide was measured. Neutrophils were prepared from fresh rat blood and diluted with phosphate-buffered saline (PBS) and adjusted to a concentration of 10⁷ cells per ml. The purity of the preparation was 95% or higher. The test sample was pre-incubated with a cell suspension at 37° C. for 15 minutes in wells of a 96 well culture plate. Then catalase and WST-1 dye reagent were added to each incubate (including controls). Each was then activated with phorbol myristate acetate (100 ng/ml). After incubation for 1 hr at 37° C. the reaction was stopped and the level of superoxide produced from oxidation of the dye WST by the superoxide in each well of the plate determined calorimetrically at 450 nm. As a positive control, triplicate wells with aspirin (100 μg/ml) were assayed. Each sample was assessed at three different concentrations.

The effect on the production of superoxide by activated neutrophils is summarised in Table 4.

TABLE 4
Effect on Superoxide Production by Activated Neutrophils

| | | Absorbance | |
|---|---|---|---|
| | Dilution | units | % Inhibition |
| Hoki Liver Oil | 1:1000 | 0.3265 | 44.00 |
| | 1:2000 | 0.3595 | 38.34 |
| | 1:4000 | 0.3570 | 38.77 |
| Fraction 1 | 1:1000 | 0.5010 | 14.07 |
| | 1:2000 | 0.5205 | 10.72 |
| | 1:4000 | 0.5900 | 1.20 stimul |
| Fraction 2 | 1:1000 | 0.4475 | 23.24 |
| | 1:2000 | 0.4820 | 17.32 |
| | 1:4000 | 0.5315 | 8.83 |
| Fraction 3 | 1:1000 | 0.2940 | 49.57 |
| | 1:2000 | 0.5260 | 9.78 |
| | 1:4000 | 0.5150 | 11.66 |
| Fraction 4 | 1:1000 | 0.2455 | 57.89 |
| | 1:2000 | 0.2290 | 60.72 |
| | 1:4000 | 0.3480 | 40.31 |
| Control | | 0.5830 | |
| Aspirin | 100 μg/ml | 0.3095 | 46.91 |

The effect of the unfractionated oil is surprisingly very similar at all three concentrations. It is approximately the same as for a 100 μg/ml solution of aspirin. However, both Fractions 1 and 2 exhibit very little inhibitory activity. Fraction 3 shows considerable activity at the 1:1000 dilution, but this is largely lost when diluted two fold. Fraction 4, however, is quite active at all three dilutions. Inhibition is approximately the same at 1:1000 and 1:2000, and only shows a slight decrease at 1:4000. The effect of fractionation has been to concentrate most of the anti-inflammatory activity in Fraction 4.

(b) Cyclooxygenase (COX)-1 and -2 Inhibition

Fresh blood samples were obtained from a rat. The assays of COX-1 and COX-2 activities were performed according the methods described by Riendeau et al. in Brit J Pharmacol (1997) 121; 105-117. The COX-1 activity in the blood was assessed by measuring the production of thromboxane B₂ (TXB₂) and the COX-2 activity was determined by measuring prostaglandin E₂ (PGE₂) following stimulation with lipopolysaccharide. These markers were both determined using ELISA kits purchased from R & D Systems (Minneapolis, USA).

Unfractionated oil was tested for its ability to modulate the activity of COX-1 and COX-2 enzymes. 3 μl of oil was added to 500 μl of each incubation mixture. 26.84% inhibition was observed for COX-1 and 50.97% for COX-2.

In view of the role of COX-2 activity in modifying inflammatory responses, the effect of the fractions of hoki liver oil on the activity of this enzyme was determined and is summarised in Table 5. This is measured as the concentration of prostaglandin E₂ (PGE₂).

TABLE 5
Effect of Fractions of Hoki Liver Oil on Cox-2 Activity

| | | PGE₂ Conc | % |
|---|---|---|---|
| | Final Conc | (pg/ml) | of Control |
| Hoki Liver Oil | 0.04% | 515 | 66.8 |
| Fraction 1 | 0.04% | 1219 | 158.1 |
| Fraction 2 | 0.04% | 1396 | 181.1 |
| Fraction 3 | 0.04% | 557 | 72.2 |
| Fraction 4 | 0.04% | 1971 | 255.6 |
| Control (+LPS) | | 771 | |
| Inhibitor (LPS + | | 492 | 63.8 |
| Indomethacin) | | | |

The unfractionated oil (0.04%) produced a 33% inhibition of the activity. The same concentration of Fraction 3 produced about the same level of antagonism. However, the other three fractions were quite stimulatory, particularly Fraction 4.

Increases in COX-2 activity may be associated with an inflammatory response in a wide range of conditions. A preparation that is inhibitory may therefore have potential as an anti-inflammatory agent.

(c) Macrophage Assay

A diluent composed of RPMI culture media containing 10% foetal calf serum (FCS) was prepared. A density gradient barrier was prepared by mixing a working solution [Optiprep: diluent, 2:1, v/v] with further diluent in a ratio of 2.3 to 5.0. The density of 1.076 g/ml is required for isolating rat monocytes. Freshly isolated rat blood was cooled to 4° C. 5 ml of the density gradient was layered over 5 ml of the blood and 5 ml of diluent was then layered gently on top. After centrifuging at 700 g for 30 min at 4° C., the monocytes were collected from the top of the 1.076 g/ml layer and diluted with 2 volumes of diluent and the pellet was gently resuspended in Hanks Balanced Salt Solution (HBSS). The cell concentration and the relative purity were determined. 500 μl Aliquots of cell suspension were used. Aliquots (10 μl) of either the test solution or indomethacin (inhibitor) were added to appropriate tubes and the cells incubated at 37° C. for 1 hour. 10 μl of LPS (5 mg/ml) was then added to all tubes, except the control, to give a final concentration of LPS of approximately 20 μg/ml. The cells were incubated overnight at 37° C. and then centrifuged at 12,000 g (4° C.) for 5 min. The supernatants were collected and aliquots of these were then assayed in duplicate for the concentration of nitric oxide (NO) by the Griess reagent procedure using a kit (Sigma, St Louis, USA).

The effect on the conversion of monocytes to macrophages as determined by the production of nitric oxide is summarised in Table 6.

TABLE 6
Effect on the Conversion of Monocytes to Macrophages

| | | Absorbance | |
|---|---|---|---|
| | Dilution | units | % Inhibition |
| Hoki Liver Oil | 1:4000 | 0.754 | 73.53 |
| | 1:5000 | 2.457 | 13.72 |
| | 1:10000 | 2.443 | 14.22 |
| Fraction 1 | 1:4000 | 0.934 | 67.21 |
| | 1:5000 | 2.540 | 10.83 |
| | 1:10000 | 2.811 | 1.30 |
| Fraction 2 | 1:4000 | 0.029 | 98.98 |
| | 1:5000 | 1.904 | 33.16 |
| | 1:10000 | 2.063 | 27.57 |
| Fraction 3 | 1:4000 | 0.336 | 88.20 |
| | 1:5000 | 0.573 | 79.87 |
| | 1:10000 | 1.149 | 59.67 |
| Fraction 4 | 1:4000 | 0.138 | 95.15 |
| | 1:5000 | 0.284 | 90.03 |
| | 1:10000 | 0.875 | 69.27 |
| Control | | 2.848 | |
| L-NMMA | 5 μM | 0.710 | 75.07 |
| | 2.5 μM | 1.568 | 44.94 |

Unfractionated oil was inhibitory of the nitric oxide production at 1:4000 dilution. It had an activity equivalent to 5 μM L-NMMA. At lower concentrations, it had virtually no effect. Fraction 1 behaved very similarly to unfractionated oil. However, at 1:4000, Fraction 2 was completely inhibitory. At 1:5000 and 1:10000, Fraction 2 was approximately 30% inhibitory, which is considerably more antagonistic than the same concentration of Fraction 1.

Fractions 3 and 4 were strongly inhibitory at both 1:4000 and 1:5000 dilutions. At 1:10000 dilution, both had lost some inhibitory activity but each was at least 60% inhibitory. Fraction 4 was slightly stronger than Fraction 3. These activities show that most of the anti-inflammatory activity was concentrated in these fractions. At this dilution the effect on NO production is equivalent to about 4 μM L-NMMA.

Although the invention has been described by way of example, it should be appreciated that variations and modifications may be made without departing from the scope of the claims. Furthermore, where known equivalents exist to specific features, such equivalents are incorporated as if specifically referred in this specification.

### INDUSTRIAL APPLICABILITY

The compositions of the invention containing hoki liver oil, or fractions thereof, are useful for treating a variety of diseases or disorders associated with angiogenesis and inflammation, including cancer, retinopathy, macula degeneration, arthritis, psoriasis, inflammations of joints, lungs, skin, or gut, infections and cardiac conditions. Certain oil fractions are potent stimulants of the immune system, and are useful for treating diseases or disorders such as cancer, microbial infection, or toxicity, whereas other fractions are potent suppressants of the immune system are therefore useful for the treatment of autoimmune diseases or for preventing rejection following organ transplantation

1.-27. (canceled)

28. A composition containing hoki liver oil, or a fraction thereof, and a suitable carrier, diluent or excipient, which composition inhibits angiogenesis or inflammation, or modulates the immune system.

29. A composition as claimed in claim 28 which inhibits both angiogenesis and inflammation.

30. A composition as claimed in claim 29 which additionally modulates the immune system.

31. A composition as claimed in claim 28 which contains a hoki liver oil fraction.

32. A composition as claimed in claim 31 where the fraction contains free fatty acids.

33. A composition as claimed in claim 32 where the free fatty acids are long chain free fatty acids of at least 20 carbon atoms.

34. A composition as claimed in claim 31 where the fraction is enriched in at least one omega-3 polyunsaturated fatty acid (or ester thereof) relative to hoki liver oil.

35. A composition as claimed in claim 31 where the fraction contains at least 5% (by weight of total free fatty acids) of eicosapentaenoic acid (EPA).

36. A composition as claimed in claim 31 where the fraction contains at least 15% (by weight of total free fatty acids) of docosahexaenoic acid (DHA).

37. A composition as claimed in claim 31 where the fraction is enriched in triglycerides relative to hoki liver oil.

38. A composition as claimed in claim 37 where the fraction contains both diglycerides and triglycerides.

39. A composition as claimed in claim 31 where the fraction contains no phospholipids.

40. A method of preventing or treating a disease or disorder associated with angiogenesis or inflammation, or modulation of the immune system, by administering to a human or non-human animal a therapeutically effective amount of hoki liver oil, or a fraction thereof.

41. A method as claimed in claim 40 where the angiogenesis is associated with cancer, retinopathy, macula degeneration, arthritis or psoriasis.

42. A method as claimed in claim 41 where the cancer is a solid tumour cancer selected from the group comprising melanoma, prostate cancer, brain cancer, colon cancer, lung cancer, and breast cancer.

43. A method as claimed in claim 41 where the arthritis is rheumatoid arthritis or osteoarthritis.

44. A method as claimed in claim 40 where the inflammation is associated with inflammations of joints, lungs, skin, or gut, or with infections or cardiac conditions.

45. A method as claimed in claim 40 where the modulation of the immune system is stimulation of the immune system for the treatment of cancer, microbial infection, or toxicity.

46. A method as claimed in claim 40 where the modulation of the immune system is suppression of the immune system for preventing rejection following organ transplantation.

47. The use of hoki liver oil, or a fraction thereof, as an anti-angiogenesis agent, or an anti-inflammatory agent, or as an immuno-modulation agent.
